(19) United States
(12) Patent Application Publication
(10) Pub. No.: US 2002/0160048 A1
(43) Pub. Date: Oct. 31, 2002

(54) Medical formulation containing a muscarinic agonist

(76) Inventors: Karoline Bechtold-Peters, Biberach (DE); Thomas Friedl, Ochsenhausen (DE); Michael Walz, Bingen (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/054,592

(22) Filed: Jan. 22, 2002

Related U.S. Application Data
(60) Provisional application No. 60/281,345, filed on Apr. 4, 2001.

(30) Foreign Application Priority Data
Feb. 15, 2001 (DE) 101 06 971.5

(51) Int. Cl. A61K 9/30

(52) U.S. Cl. 424/475

(57) ABSTRACT

A film-coated tablet containing talsaclidine comprising:

(a) a core comprising talsaclidine or a physiologically acceptable acid addition salt thereof; and

(b) a film coating,

wherein the film coating envelops the core.

### RELATED APPLICATIONS

Benefit under 35 U.S.C. § 119(e) of prior provisional application Serial No. 60/281,345, filed Apr. 4, 2001, is hereby claimed.

### FIELD OF THE INVENTION

The invention relates to a new pharmaceutical formulation containing the muscarinic agonist talsaclidine and processes for preparing it.

### BACKGROUND OF THE INVENTION

Talsaclidine (Wa12014), being a muscarinic agonist, is a pharmacologically valuable compound. Muscarinic agonists may be of great therapeutic benefit in the treatment of Alzheimer's disease, for example. Talsaclidine 1′ has the following chemical structure:

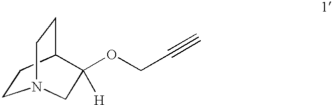

The aim of the present invention is to develop a pharmaceutical formulation for oral administration which releases the active substance talsaclidine relatively rapidly and completely. A further aim of the present invention is to provide a formulation which is characterized by ease of handling during the preparation process and can therefore be reproducibly manufactured on an industrial scale while maintaining a high quality.

### DETAILED DESCRIPTION OF THE INVENTION

The above objectives can be achieved by the formulation described in detail hereinafter.

The present invention relates to a tablet containing talsaclidine 1′, characterized in that it consists of a core containing the active substance talsaclidine and a film coating enclosing this core. The tablet according to the invention may also be termed a film-coated tablet within the scope of the present invention.

The active substance talsaclidine is preferably present in the formulation according to the invention in the form of a physiologically acceptable acid addition salt 1. The term physiologically acceptable acid addition salts according to the invention denotes pharmaceutically acceptable salts selected from among the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid. If desired, mixtures of the above acids may also be used to prepare the salts. According to the invention the preferred salts of talsaclidine are selected from among the hydrochloride, hydrobromide, sulfate, phosphate, fumarate, and methanesulfonate. Most preferably, the salts are selected from among the hydrochloride, hydrobromide, and fumarate, while talsaclidine fumarate is most important according to the invention. The active substance may optionally be in the form of a hydrate. Preferably, however, according to the invention, the talsaclidine is added in an anhydrous form. The active substance is preferably used in crystalline, unground form or after being ground in a pinned disc mill, but preferably in unground form. More preferably, the active substance is used in unground form with a particle size distribution within the following limits: D₁₀≦20 μm, D₅₀ is 10 μm to 80 μm, and D₉₀≦300 μm. Most preferably, the particle size distribution of the active substance used in the formulation according to the invention is in the following ranges: D₁₀≦5 μm to 15 μm, D₅₀ is 25 μm to 75 μm, D₉₀≦275 μm. The numerical values given above for D₁₀, D₅₀, and D₉₀ in μm (microns) are the particle size ranges within which a through total of 10 vol. %, 50 vol. %, or 90 vol. % of the measured particles (cumulative volume distribution) is achieved. These values were determined by the laser diffractometry method, in the present instance particularly using a so-called dry dispersion at a 2 bar dispersion pressure and a focal length f=500 mm, e.g., using a Sympatec/RODOS apparatus. This method is known in the prior art.

Where reference is made to salts of talsaclidine within the scope of the present invention, this is indicated by the numeral 1. Any explicit reference to the base talsaclidine, on the other hand, is indicated by the use of the numeral 1′. Based on the total mass of the core of the film-coated tablets according to the invention talsaclidine 1′ is present according to the invention in amounts of 0.5 wt. % to 25 wt. %, preferably 0.7 wt. % to 20 wt. %, particularly preferably about 0.9 wt. % to 15 wt. %. Particularly preferably, the proportion of 1′ is between 9 wt. % and 14 wt. % based on the total mass of the core. If talsaclidine is used for example in the form of the preferred salt 1 talsaclidine fumarate according to the invention in the formulation according to the invention, the proportion of 1 based on the total mass of the core of the film-coated tablets according to the invention is between about 0.85 wt. % and 43 wt. %, preferably between about 1.2 wt. % and 34 wt. %, particularly preferably between about 1.5 wt. % and 26 wt. %. Particularly preferably the proportion of 1 in the case of talsaclidine fumarate is between about 15 wt. % and 24 wt. % based on the total mass of the core.

The core of the pharmaceutical formulation according to the invention contains, in addition to the active substance, at least one excipient as filler/dry binder.

Within the scope of the present invention modified lactose, particularly spray-dried lactose is of particular importance as the excipient. This excipient has proved particularly advantageous in the formulation according to the invention. A preferred aspect of the present invention thus relates to a film-coated tablet containing talsaclidine which contains in its core, in addition to the active substance, modified lactose, particularly spray-dried lactose, particularly preferably spray-dried lactose monohydrate as excipient.

By spray-dried lactose is meant lactose produced by spray agglomeration when spray-drying a suspension of a-lactose monohydrate crystals in an aqueous lactose solution. The spray-drying process results in a free-flowing powder with a granulometry suitable for direct tabletting (for example, 80% to 100% <250 μm) and an amorphous content of, for example, 5% to 25%, which is responsible for the high binding power of spray-dried lactose.

According to the invention the weight ratio between the components contained in the core of the film-coated tablet, namely modified lactose, preferably spray-dried lactose, to active substance 1′ is in the range from about 1:1 to about 70:1. Preferably, the ratio of modified, preferably spray-dried lactose to 1′ is in the range from about 1.5:1 to about 35:1, particularly preferably in a range from about 2:1 to about 8:1. Preferably, the proportion by weight of modified, preferably spray-dried lactose based on the total mass of the core of the film-coated tablet according to the invention is in a range from about 20 wt. % to 70 wt. %, preferably between about 30 wt. % to 60 wt. %.

The core of the film-coated tablet according to the invention may also contain, in addition to modified, preferably spray-dried lactose and active substance, other excipients or fillers. According to the invention it is preferable to use those compounds which can act as dry binders. Preferred dry binders according to the invention are selected from among powdered cellulose, microcrystalline cellulose, starch, povidone, cellulose derivatives and mixtures of these compounds. Preferred binders are powdered cellulose and /or microcrystalline cellulose, particularly preferably microcrystalline cellulose. If one of the abovementioned binders is added to the formulation according to the invention, the weight ratio of modified, preferably spray-dried lactose to binder is preferably about 5:1 to about 1:4, preferably about 4:1 to about 1:3, particularly preferably about 3:1 to 1:2. It is particularly preferable according to the invention if the weight ratio of spray-dried lactose to binder is in the range from about 2:1 to about 1:1.

The core of the film-coated tablet according to the invention may also contain disintegrants in addition to the ingredients mentioned above. Within the scope of the present invention these disintegrants may optionally also be known as breakdown agents. These are preferably selected according to the invention from among sodium starch glycolate, crospovidone, croscarmellose, sodium-carboxymethylcellulose, dried corn starch and mixtures thereof. Particularly preferably, within the scope of the present invention, sodium starch glycolate, crospovidone and croscarmellose, preferably sodium starch glycolate, are used. If the abovementioned disintegrants are used, the amount by weight used based on the total mass of the core of the film-coated tablet according to the invention is preferably in a range from about 1 wt. % to 10 wt. %, particularly preferably about 3 wt. % to 8 wt. %.

The core of the film-coated tablet according to the invention may also contain flow regulators as additional ingredients. Flow regulators within the scope of the present invention include, for example, silicon dioxide, talc and magnesium stearate. According to the invention silicon dioxide is preferably used, particularly preferably in colloidal, highly dispersed form. If the abovementioned flow regulators are used, the amount by weight thereof based on the total mass of the core of the film-coated tablet according to the invention is preferably in a range from about 0.1 wt. % to 5 wt. %, preferably about 0.3 wt. % to 2 wt. %, particularly preferably between 0.4 wt. % and 1.5 wt. %.

The core of the film-coated tablet according to the invention may also contain flow agents, lubricants and mould release agents as further ingredients. These include, for example, within the scope of the present invention, stearic acid, magnesium stearate, sodium stearyl fumarate, glycerol tribehenate and mixtures thereof. According to the invention, stearic acid and magnesium stearate are preferably used. The amount by weight based on the total mass of the core of the film-coated tablet according to the invention is preferably in a range from about 0.1 wt. % to 5 wt. %, preferably about 0.5 wt. % to 3 wt. %, particularly preferably about 1 wt. % to 2 wt. %. Particularly good release from the mould in the production of the film-coated tablets according to the invention is achieved when magnesium stearate is used in an amount of at least 1.0 wt. %, preferably about 1.5 wt. %.

The film or film coating enveloping the core of the film-coated tablets according to the invention contains as essential ingredient a film-forming agent selected from among hydroxypropylmethylcellulose, hydroxypropylcellulose, methylcellulose, hydroxymethylcellulose, hydroxyethylcellulose and poly(ethylacrylate) methylmethacrylate, the latter in the form of EUDRAGIT® NE 30 D, for example. Alternatively, EUDRAGIT® RL 30 D or EUDRAGIT® E 12.5 may be used, for example. The above ingredients may optionally also be used in the form of mixtures thereof. Preferred film-forming agents are hydroxypropylmethylcellulose, hydroxypropylcellulose, hydroxymethylcellulose and hydroxyethylcellulose, of which hydroxypropylmethylcellulose and hydroxypropylcellulose are particularly preferred as film-forming agents according to the invention. The abovementioned film-forming agents may be used on their own or in the form of the mixtures thereof. If only one of the abovementioned film-forming agents is used, hydroxypropylmethylcellulose is of particular importance in this context within the scope of the present invention. The amount by weight of film-forming agents based on the total mass of the film coating of the film-coated tablet according to the invention is preferably in a range from about 20 wt. % to 95 wt. %, preferably 30 wt. % to 90 wt. %.

The film coating may contain, as further ingredients, emulsifiers and plasticizers such as, for example, polyethyleneglycol, glycerol and propyleneglycol, optionally in the form of the mixtures thereof. Preferably, polyethyleneglycols are used as plasticizers. Without restricting the subject matter of the invention thereto, polyethyleneglycol 400 and polyethyleneglycol 6000 are examples of particularly preferred polyethyleneglycols. The amount of plasticizer by weight based on the total mass of the film coating of the film-coated tablet according to the invention is preferably in a range from about 1 to 30 wt. %, preferably 3 wt. % to 25 wt. %, particularly preferably 5 wt. % to 15 wt. %.

The film coating of the film-coated tablet according to the invention may also contain colored pigments and pigmenting excipients. Iron oxide, titanium dioxide, talc, and mixtures thereof may be mentioned by way of example.

The following procedure may be used, for example, to prepare the film-coated tablet according to the invention.

In a suitable free-fall mixer the excipient spray-dried lactose is mixed with the active substance 1. The active substance premix obtained therefrom is then mixed in a suitable screening machine. This reduces the proportion of coarser particles of active substance which may possibly go into the manufacturing process. Depending on the particle size of the compound 1 used, the preparation of this premix with the subsequent screening process may also be avoided.

In another step of the process, more excipient is added to this active substance premix. As well as spray-dried lactose one or more other excipients may also be added. Moreover, during this step of the process, other ingredients of the formulation such as disintegrants and flow regulators may optionally also be added. After the mixing process has finished the mixture obtained is screened again. This intermediate screening is the essential step for obtaining a uniformity of the contents of the mixture in conformity with the Pharmacopoeia. The flow agent, lubricant and mould release agent are then added to this screened active substance mixture.

The mixture of active substance and excipient thus obtained is then compressed in a suitable tablet press to form the film-coated tablet cores according to the invention. The compressing force should be kept within a range from 5 kN to 15 kN, preferably 8 kN to 12 kN, for example, in the case of tablet cores which contain 36 mg of talsaclidine 1′. Higher compressing forces may lead to tablets with a delayed release of active substance. Lower compressing forces may lead to tablets which are mechanically unstable.

The tablet cores may take various forms, of which round biplanar or biconvex and oval or oblong shapes are preferred.

In order to prepare the film coating suspension the essential and optional ingredients of the film coating are taken up in a suitable solvent. According to the invention water is preferably used as the solvent. When water is used as solvent, the ingredients of the film coating are partly in dispersed form.

After the coating suspension is finished the tablet cores obtained previously are coated with the desired film in a suitable coating apparatus analogously to coating methods known in the art.

The Examples that follow serve to illustrate some formulations according to the invention. They are intended solely as possible methods given by way of example, without restricting the invention to their content.

EXAMPLE 1

| Tablet | | Amount |
|---|---|---|
| Portion | Ingredient | (mg) |
| Core | talsaclidine fumarate | 61.287 |
| | lactose monohydrate (spray-dried) | 99.363 |
| | microcrystalline cellulose | 90.450 |
| | sodium starch glycolate | 13.500 |
| | highly dispersed silicon dioxide | 1.350 |
| | magnesium stearate | 4.050 |
| | Total (core) | 270.000 |
| Film | hydroxypropylmethylcellulose | 3.500 |
| Coating | polyethyleneglycol 400 | 0.350 |
| | titanium dioxide | 1.750 |
| | talc | 1.358 |
| | iron oxide (yellow) | 0.042 |
| | Total (film coating) | 7.000 |
| | Total (film-coated tablet) | 277.000 |

EXAMPLE 2

| Tablet | | Amount |
|---|---|---|
| Portion | Ingredient | (mg) |
| Core | talsaclidine fumarate | 5.11 |
| | lactose monohydrate (spray-dried) | 209.09 |
| | microcrystalline cellulose | 102.00 |
| | sodium starch glycolate | 17.00 |
| | highly dispersed silicon dioxide | 3.40 |
| | stearic acid | 3.40 |
| | Total (core) | 340.00 |
| Film | hydroxypropylmethylcellulose | 1.1416 |
| Coating | polyethyleneglycol 6000 | 1.4269 |
| | titanium dioxide | 1.5696 |
| | talc | 4.5662 |
| | methacrylic acid copolymer EUDRAGIT ® | 1.2843 |
| | Total (film coating) | 10.000 |
| | Total (film-coated tablet) | 350.00 |

EXAMPLE 3

| Tablet | | Amount |
|---|---|---|
| Portion | Ingredient | (mg) |
| Core | talsaclidine fumarate | 10.21 |
| | lactose monohydrate (spray-dried) | 203.99 |
| | microcrystalline cellulose | 102.00 |
| | sodium starch glycolate | 17.00 |
| | highly dispersed silicon dioxide | 3.40 |
| | stearic acid | 3.40 |
| | Total (core) | 340.00 |
| Film | Film Coating has the Same | |
| Coating | Composition as Example 2 | |
| | Total (film coating) | 10.000 |
| | Total (film-coated tablet) | 350.00 |

EXAMPLE 4

| Tablet | | Amount |
|---|---|---|
| Portion | Ingredient | (mg) |
| Core | talsaclidine fumarate | 20.43 |
| | lactose monohydrate (spray-dried) | 193.77 |
| | microcrystalline cellulose | 102.00 |
| | sodium starch glycolate | 17.00 |
| | highly dispersed silicon dioxide | 3.40 |
| | stearic acid | 3.40 |
| | Total (core) | 340.00 |
| Film | Film Coating has the Same | |
| Coating | Composition as Example 2 | |
| | Total (film coating) | 10.000 |
| | Total (film-coated tablet) | 350.00 |

EXAMPLE 5

| Tablet | | Amount |
|---|---|---|
| Portion | Ingredient | (mg) |
| Core | talsaclidine fumarate | 40.86 |
| | lactose monohydrate (spray-dried) | 173.34 |
| | microcrystalline cellulose | 102.00 |
| | sodium starch glycolate | 17.00 |
| | highly dispersed silicon dioxide | 3.40 |
| | stearic acid | 3.40 |
| | Total (core) | 340.00 |
| Film | Film Coating has the Same | |
| Coating | Composition as Example 2 | |
| | Total (film coating) | 10.000 |
| | Total (film-coated tablet) | 350.00 |

EXAMPLE 6

| Tablet | | Amount |
|---|---|---|
| Portion | Ingredient | (mg) |
| Core | talsaclidine fumarate | 51.07 |
| | lactose monohydrate (spray-dried) | 163.13 |
| | microcrystalline cellulose | 102.00 |
| | sodium starch glycolate | 17.00 |
| | highly dispersed silicon dioxide | 3.40 |
| | stearic acid | 3.40 |
| | Total (core) | 340.00 |
| Film | Film Coating has the Same | |
| Coating | Composition as Example 2 | |
| | Total (film coating) | 10.000 |
| | Total (film-coated tablet) | 350.00 |

EXAMPLE 7

| Tablet | | Amount |
|---|---|---|
| Portion | Ingredient | (mg) |
| Core | talsaclidine fumarate | 61.29 |
| | lactose monohydrate (spray-dried) | 152.91 |
| | microcrystalline cellulose | 102.00 |
| | sodium starch glycolate | 17.00 |
| | highly dispersed silicon dioxide | 3.40 |
| | stearic acid | 3.40 |
| | Total (core) 340.00 | |
| Film | Film Coating has the Same | |
| Coating | Composition as Example 2 | |
| | Total (film coating) | 10.000 |
| | Total (film-coated tablet) | 350.00 |

EXAMPLE 8

| Tablet | | Amount |
|---|---|---|
| Portion | Ingredient | (mg) |
| Core | talsaclidine fumarate | 81.72 |
| | lactose monohydrate (spray-dried) | 132.48 |
| | microcrystalline cellulose | 102.00 |
| | sodium starch glycolate | 17.00 |
| | highly dispersed silicon dioxide | 3.40 |
| | stearic acid | 3.40 |
| | Total (core) | 340.00 |
| Film | Film Coating has the Same | |
| Coating | Composition as Example 2 | |
| | Total (film coating) | 10.000 |
| | Total (film-coated tablet) | 350.00 |

EXAMPLE 9

| Tablet | | Amount |
|---|---|---|
| Portion | Ingredient | (mg) |
| Core | talsaclidine fumarate | 40.86 |
| | lactose monohydrate (spray-dried) | 66.24 |
| | microcrystalline cellulose | 60.30 |
| | sodium starch glycolate | 9.00 |
| | highly dispersed silicon dioxide | 0.90 |
| | stearic acid | 2.70 |
| | Total (core) | 180.00 |
| Film | Film Coating has the Same | |
| Coating | Composition as Example 1 | |
| | Total (film coating) | 5.000 |
| | Total (film-coated tablet) | 350.00 |

We claim:

1. A film-coated tablet containing talsaclidine comprising:
(a) a core comprising talsaclidine or a physiologically acceptable acid addition salt thereof; and
(b) a film coating,
wherein the film coating envelops the core.

2. The tablet according to claim 1, wherein the core further comprises a modified lactose excipient.

3. The tablet according to claim 2, wherein the modified lactose excipient is spray-dried lactose.

4. The tablet according to claim 1, wherein the talsaclidine is in the form of an acid addition salt selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid.

5. The tablet according to claim 1, wherein the talsaclidine in the core is present in an amount of from 0.5 wt. % to 25 wt. % of the total mass of the core.

6. The tablet according to claim 1, wherein the talsaclidine in the core is present in an amount of from 0.7 wt. % to 20 wt. % of the total mass of the core.

7. The tablet according to claim 2, wherein the weight ratio between the modified lactose excipient to talsaclidine is in a range from about 1:1 to about 70:1.

8. The tablet according to claim 2, wherein the weight ratio between the modified lactose excipient to talsaclidine is in a range from about 1.5:1 to about 35:1.

9. The tablet according to claim 2, wherein the core further comprises a dry binder.

10. The tablet according to claim 9, wherein the weight ratio of the modified lactose excipient to dry binder is in the range from about 5:1 to about 1:4.

11. The tablet according to claim 10, wherein the weight ratio of the modified lactose excipient to dry binder is in the range from about 4:1 to about 1:3.

12. The tablet according to claim 2, wherein the core further comprises a disintegrant.

13. The tablet according to claim 12, wherein the disintegrant in the core is present in an amount of from about 1 wt. % to about 10 wt. % of the total mass of the core.

14. The tablet according to claim 1, wherein the core further comprises a flow regulator.

15. The tablet according to claim 14, wherein the flow regulator in the core is present in an amount of from about 0.1 wt. % to about 5 wt. % of the total mass of the core.

16. The tablet according to claim 1, wherein the core further comprises a flow agent, lubricant, or mould release agent.

17. The tablet according to claim 16, wherein the flow agent, lubricant, and mould release agent in the core are present in an amount of from about 0.1 wt. % to about 5 wt. % of the total mass of the core.

18. The tablet according to claim 1, wherein the film coating comprises a film-forming agent selected from the group consisting of: hydroxymethylcellulose, hydroxyethylcellulose, hydroxypropylcellulose, hydroxypropylmethylcellulose, methylcellulose, and poly(ethylacrylate) methylmethacrylate.

19. The tablet according to claim 1, wherein the film coating comprises a film-forming agent selected from the group consisting of: EUDRAGIT® NE 30 D, EUDRAGIT® RL 30 D, and EUDRAGIT® E.

20. The tablet according to claim 18, wherein the film-forming agents in the film coating are present in an amount of from about 20 wt. % to about 95 wt. % of the total mass of the film coating.

21. The tablet according to claim 18, wherein the film coating further comprises an emulsifier or a plasticizer.

22. The tablet according to claim 18, wherein the film coating further comprises a plasticizer.

23. The tablet according to claim 22, wherein the plasticizers in the film coating are present in an amount of from about 1 wt. % to about 30 wt. % of the total mass of the film coating.
